(19) United States
(12) Patent Application Publication
(10) Pub. No.: US 2023/0096820 A1
(43) Pub. Date: Mar. 30, 2023

(54) METHODS, SYSTEMS, AND COMPUTER PROGRAM PRODUCTS FOR AUTOMATICALLY PROCESSING A CLINICAL RECORD FOR A PATIENT TO DETECT PROTECTED HEALTH INFORMATION (PHI) VIOLATIONS

(71) Applicant: Change Healthcare Holdings LLC, Nashville, TN (US)

(72) Inventor: Alex Londeree, Austin, TX (US)

(21) Appl. No.: 17/449,334

(22) Filed: Sep. 29, 2021

(51) Int. Cl. G16H 10/60; G16H 50/20; G16H 50/70; G06N 5/02; G06F 16/901; G06K 9/00

(52) U.S. Cl. CPC G16H 10/60; G16H 50/20; G16H 50/70; G06N 5/022; G06F 16/9024; G06K 9/00442; G16H 40/20

(57) ABSTRACT

A method includes receiving a record including at least one page and containing clinical information associated with a first patient; receiving respective first patient identification values for one or more patient identification parameters corresponding to the first patient; automatically processing the record to identify first example instances referencing the patient identification parameters including values therefor; automatically processing the record to identify second example instances of the first patient identification values; automatically processing the record to determine whether any of the at least one page contained therein cannot be semantically linked to another one of the at least one page in the record; and assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.

### FIELD

The present inventive concepts relate generally to health care systems and services and, more particularly, to managing clinical records for patients to reduce privacy violations.

### BACKGROUND

Health care service providers record clinical information associated with patients under their care in clinical charts, which are typically stored as electronic health records. These charts or health records may pass through many different health care professionals and other entities in the process of providing care to the patients. Clinical charts or health records for multiple patients may be consolidated for batch transfer between entities. Once a transfer is complete, the consolidated batch document is split back into the individual patient charts or records. This consolidation and subsequent splitting may not always be performed accurately resulting in the charts or records for multiple patients remaining grouped together. Other times one or more pages from one patient's medical chart or record may end up in another patient's medical chart or record. Considering the size and scope of many health care systems, there are numerous ways for patient records to become intermingled. If a patient's chart or record refers to another patient, however, this could represent a protected health information (PHI) violation, if the party accessing the chart or record for any of a variety of different administrative tasks, such as data entry, medical coding, and/or auditing, does not have the rights to access the second patient's chart or record. Moreover, the co-mingling of chart or record information between patients could lead to errors in patient care by medical professionals.

### SUMMARY

According to some embodiments of the inventive concept, a method comprises: receiving a record comprising at least one page and containing clinical information associated with a first patient; receiving respective first patient identification values for one or more patient identification parameters corresponding to the first patient; automatically processing the record to identify first example instances referencing the patient identification parameters including values therefor; automatically processing the record to identify second example instances of the first patient identification values; automatically processing the record to determine whether any of the at least one page contained therein cannot be semantically linked to another one of the at least one page in the record; and assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.

In other embodiments, the method further comprises converting the record into a text record; wherein automatically processing the record to identify first example instances, automatically processing the record to identify second example instances, and automatically processing the record to determine whether any of the at least one page contained therein cannot be semantically linked to another one of the at least one page in the record, comprises: processing the text record to identify first example instances, processing the text record to identify second example instances, and processing the text record to determine whether any of the at least one page contained therein cannot be semantically linked to another one of the at least one page in the text record.

In still other embodiments, converting the record into the text record comprises converting the record into the text record using optical character recognition (OCR).

In still other embodiments, the patient identification parameters comprise: patient demographic parameters, pharmacy information parameters, diagnosis code parameters, and provider encounter parameters.

In still other embodiments the patient demographic parameters comprise: patient name, patient address, patient mobile phone number, patient home phone number, patient work phone number, patient guardian name, patient guardian address, patient guardian mobile phone number, patient guardian home phone number, patient guardian work phone number, patient gender, patient date of birth, patient social security number, patient provider identification number, patient marital status, patient email, patient preferred language, patient race, patient primary care physician name, patient emergency contact name, patient emergency contact phone number, patient employer name, and/or patient employer address.

In still other embodiments, the pharmacy information parameters comprise: pharmacy name, pharmacy address, and/or pharmacy phone number.

In still other embodiments, the provider encounter parameters comprise: encounter date, name of medical professional, and/or identification of service performed.

In still other embodiments, automatically processing the record to identify the first example instances referencing the patient identification parameters including the values therefor comprises: determining a Levenshtein distance between each of the first example instances referencing the patient identification parameters and the patient identification parameters, respectively.

In still other embodiments, determining the Levenshtein distance comprises: determining the Levenshtein distance using fuzzy matching, regular expression analysis, and/or language modeling.

In still other embodiments, automatically processing the record to identify the second example instances of the first patient identification values comprises: determining a Levenshtein distance between each of the second example instances of the first patient identification values and the first patient identification values, respectively.

In still other embodiments, determining the Levenshtein distance comprises: determining the Levenshtein distance using fuzzy matching, regular expression analysis, and/or language modeling.

In still other embodiments, automatically processing the record to identify the first example instances referencing the patient identification parameters including the values therefor comprises: generating a patient identification parameter extraction model based on historical records containing historical clinical information for patients in which associations are learned between the patient identification parameters and manners in which the historical clinical information is organized in the historical records.

In still other embodiments, generating the patient identification parameter extraction model comprises: using an Artificial Intelligence (AI) system to learn the associations between the patient identification parameters and the manners in which the historical clinical information is organized in the historical records.

In still other embodiments, automatically processing the record to determine whether any of the at least one page contained therein cannot be semantically linked to another one of the at least one page in the record comprises: determining whether adjacent pages in the record have sequential numbers; determining whether any of the at least one page contained in the record references a different case number than other ones of the at least one page contained in the record; and/or determining whether content between adjacent ones of the at least one page in the record is continuous.

In still other embodiments, assigning the grade to each of the at least one page indicating the degree of confidence that the record does not include clinical information associated with the second patient comprises: assigning the grade of confirmed when the page satisfies a threshold criterion that the page does not include clinical information associated with the second patient indicating the page does not need manual review; assigning the grade of suspicious when the page does not satisfy the threshold criterion and is predicted to include clinical information associated with the second patient and needs correction; and assigning the grade of unknown when a prediction whether the page includes clinical information associated with the second patient cannot be made and manual review is recommended.

In still other embodiments, assigning the grade to each of the at least one page indicating the degree of confidence that the record does not include clinical information associated with the second patient comprises: assigning a grade to the record based on the grades assigned to each of the at least one page indicating a degree of confidence that the record does not include clinical information associated with the second patient.

In some embodiments of the inventive concept, a system comprises a processor; and a memory coupled to the processor and comprising computer readable program code embodied in the memory that is executable by the processor to perform operations comprising: receiving a record comprising at least one page and containing clinical information associated with a first patient; receiving respective first patient identification values for one or more patient identification parameters corresponding to the first patient; automatically processing the record to identify first example instances referencing the patient identification parameters including values therefor; automatically processing the record to identify second example instances of the first patient identification values; automatically processing the record to determine whether any of the at least one page contained therein cannot be semantically linked to another one of the at least one page in the record; and assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.

In further embodiments, automatically processing the record to identify the first example instances referencing the patient identification parameters including the values therefor comprises: determining a Levenshtein distance between each of the first example instances referencing the patient identification parameters and the patient identification parameters, respectively; and automatically processing the record to identify the second example instances of the first patient identification values comprises: determining a Levenshtein distance between each of the second example instances of the first patient identification values and the first patient identification values, respectively.

In still further embodiments, assigning the grade to each of the at least one page indicating the degree of confidence that the record does not include clinical information associated with the second patient comprises: assigning the grade of confirmed when the page satisfies a threshold criterion that the page does not include clinical information associated with the second patient indicating the page does not need manual review; assigning the grade of suspicious when the page does not satisfy the threshold criterion and is predicted to include clinical information associated with the second patient and needs correction; and assigning the grade of unknown when a prediction whether the page includes clinical information associated with the second patient cannot be made and manual review is recommended.

In some embodiments, a computer program product comprises a non-transitory computer readable storage medium comprising computer readable program code embodied in the medium that is executable by a processor to perform operations comprising: receiving a record comprising at least one page and containing clinical information associated with a first patient; receiving respective first patient identification values for one or more patient identification parameters corresponding to the first patient; automatically processing the record to identify first example instances referencing the patient identification parameters including values therefor; automatically processing the record to identify second example instances of the first patient identification values; automatically processing the record to determine whether any of the at least one page contained therein cannot be semantically linked to another one of the at least one page in the record; and assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a protected health information (PHI) violation detection system in accordance with some embodiments of the inventive concept;

FIG. 2 is a flowchart that illustrates operations for automatically detecting a possible PHI violation in a patient clinical record in accordance with some embodiments of the inventive concept;

FIGS. 3 and 4 are block diagrams that illustrate the PHI violation detection system in accordance with some embodiments of the inventive concept;

FIGS. 5 and 6 are flowcharts that illustrate further operations for automatically detecting a possible PHI violation in a patient clinical record in accordance with further embodiments of the inventive concept;

FIG. 7 is a data processing system that may be used to implement a PHI violation detection system in accordance with some embodiments of the inventive concept; and

FIG. 8 is a block diagram that illustrates a software/hardware architecture for use in a PHI violation detection system in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

Embodiments of the inventive concept are described herein in the context of a protected health information (PHI) violation detection system that includes an artificial intelligence (AI) engine, which uses machine learning. It will be understood that embodiments of the inventive concept are not limited to a machine learning implementation of the PHI violation detection system and other types of AI systems may be used including, but not limited to, a multi-layer neural network, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons.

Some embodiments of the inventive concept stem from a realization that the handling of clinical care charts or health records for patients by different medical professionals and/or other entities may result in a patient's chart or record including one or more pages from another patient's chart or record, which could result in a PHI violation. Traditionally, such charts or records would be manually reviewed page-by-page to verify that the entire chart or record is associated with the same patient. If one or more pages are detected that correspond to another patient, then these pages are flagged and removed to ensure each patient's privacy is not violated. Embodiments of the inventive concept may provide an automated PHI violation detection system in which, in response to a request for a first patient's clinical record, for example, the first patient's clinical record may be converted into text form for processing and evaluation. The PHI violation detection system may include an extraction component in which the record is processed to identify instances that reference metadata corresponding to patient identification parameters. The extraction component may, therefore, identify examples in the record that identify any patient based on the identification parameters including the first patient to which the record belongs and any other patient whose clinical record page(s) may have found their way into the first patient's record. The PHI violation detection system may further include a matching component in which the record is processed to identify instances in which the first patient's identification values (i.e., values for one or more patient identification parameters corresponding to the first patient to which the record belongs) are found in the record. In addition to the extraction component and the matching component, the PHI violation detection system may use a linking component to identify whether any of the pages in the record are not semantically linked with one another. For example, pages with page numbers or other type of identifier that can be identified as being part of a sequence may be identified or flagged as being linked together. Pages that cannot be identified as belonging to such a linked sequence are excluded from the link flag or identification and may, therefore, be considered as potentially belonging to a different patient, chart, or record. Based on the analysis from the extraction, matching, and linking components, an assembly component may assign a grade to each of the pages in the record indicating a degree of confidence that the page does not include clinical information associated with a patient other than the first patient to which the record belongs. Based on this grade, the record may be confirmed as not needing a manual review, may be identified as likely containing one or more PHI errors that require correction, or may be identified as unknown or indeterminate with a recommendation that the page be reviewed manually. The record as a whole may be assigned a grade based on the individual grades assigned to its respective pages including a summary of the number of pages in each grade category. The automated PHI violation detection system, according to some embodiments of the inventive concept, may reduce the amount of manual review required for detecting PHI violations in patients' clinical records, which can result in significant time and expense savings due to the quantity of patient records and the potentially large number of pages in each record.

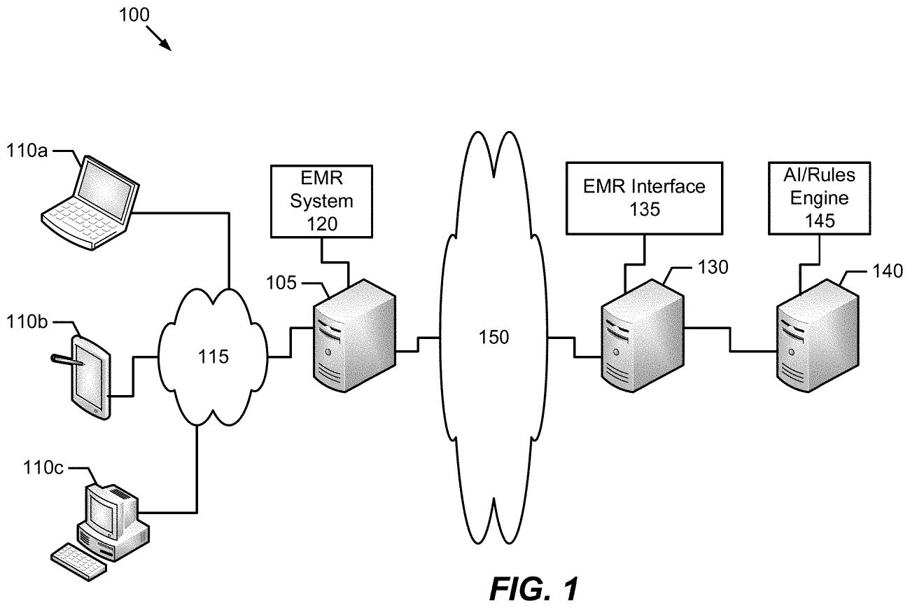

Referring to FIG. 1, a communication network 100 including PHI violation detection system, in accordance with some embodiments of the inventive concept, comprises a health care facility server 105 that is coupled to devices 110a, 110b, and 110c via a network 115. The health care facility may be any type of health care or medical facility, such as a hospital, doctor's office, specialty center (e.g., surgical center, orthopedic center, laboratory center etc.), or the like. The health care facility server 105 may be configured with an Electronic Medical Record (EMR) system module 120 to manage patient files and facilitate the entry of orders for patients via health care service providers (“providers”). Although shown as one combined system in FIG. 1, it will be understood that some health care facilities use separate systems for electronic medical record management and order entry management. The providers may use devices, such as devices 110a, 110b, and 110c to manage patients' electronic charts or records and to issue orders for the patients through the EMR system 120. An order may include, but is not limited to, a treatment, a procedure (e.g., surgical procedure, physical therapy procedure, radiologic/imaging procedure, etc.) a test, a prescription, and the like. The network 115 communicatively couples the devices 110a, 110b, and 110c to the health care facility server 105. The network 115 may comprise one or more local or wireless networks to communicate with the health care facility server 105 when the health care facility server 105 is located in or proximate to the health care facility. When the health care facility server 105 is in a remote location from the health care facility, such as part of a cloud computing system or at a central computing center, then the network 115 may include one or more wide area or global networks, such as the Internet.

According to some embodiments of the inventive concept, a PHI violation detection system may be provided to assist entities, such as providers, payors, auditors, data entry personnel, and others to automatically identify potential PHI violations in patient records. The PHI violation detection system may include a health care facility interface server 130, which includes an EMR interface system module 135 to facilitate the transfer of information between the EMR system 120, which the providers use to manage patient charts and records and issue orders, and a PHI violation detection server 140, which includes an AI/Rules engine module 145. The PHI violation detection server 140 and AI/Rules engine module 145 may be configured to receive patient records along with patient identification values for one or more patient identification parameters corresponding to the patient from the EMR system 120 by way of the health care facility interface server 130 and EMR interface module 135. The PHI violation detection server and AI/Rules engine 145 may process each page of each patient clinical record using a multi-phase approach based on an extraction component, a matching component, a linking component, and an assembly component to assign a grade to each page that is indicative of a degree of confidence that the record does not include clinical information associated with a patient other than the patient to whom the record belongs. Based on the grade, the page in the record may be confirmed as exempt from manual review to identify PHI violations, identified as needing correction of one or more likely PHI violations, or identified as needing manual review due to the automated review of the page being indeterminate with respect to the existence of a PHI violation.

It will be understood that the division of functionality described herein between the PHI violation detection server 140/AI/Rules engine module 145 and the health care facility interface server 130/EMR interface module 135 is an example. Various functionality and capabilities can be moved between the PHI violation detection server 140/AI/Rules engine module 145 and the health care facility interface server 130/EMR interface module 135 in accordance with different embodiments of the inventive concept. Moreover, in some embodiments, the PHI violation detection server 140/AI engine module 145 and the health care facility interface server 130/EMR interface module 135 may be merged as a single logical and/or physical entity.

A network 150 couples the health care facility server 105 to the health care facility interface server 130. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The PHI violation detection service provided through the health care facility interface server 130, EMR interface module 135, PHI violation detection server 140 and AI/Rules engine module 145 to automatically detect a potential PHI violation in a patient clinical record may, in some embodiments, be embodied as a cloud service. For example, entities may integrate their clinical record processing system with the PHI violation detection service and access the service as a Web service. In some embodiments, the PHI violation detection service may be implemented as a Representational State Transfer Web Service (RESTful Web service).

Although FIG. 1 illustrates an example communication network including a PHI violation detection system for detecting a potential PHI violation in a patient clinical record, it will be understood that embodiments of the inventive subject matter are not limited to such configurations, but are intended to encompass any configuration capable of carrying out the operations described herein.

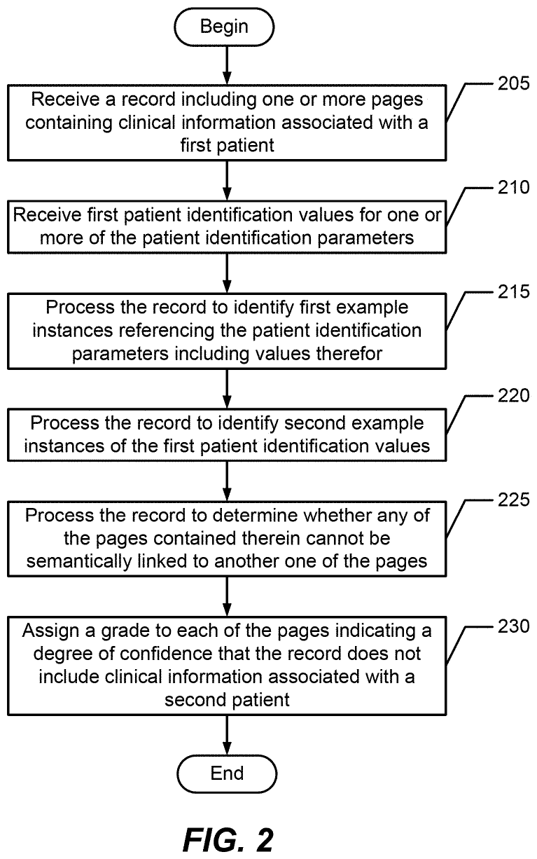

FIG. 2 is a flowchart that illustrates operations for automatically detecting possible PHI violations in a patient clinical record in accordance with some embodiments of the inventive concept. Referring now to FIG. 2, operations begin at block 205 where a record including one or more pages is received that contains clinical information associated with a first patient. The record may be received, for example, in response to a request for the clinical record for the first patient. In some embodiments, the record may be converted into a text record using, for example, optical character recognition (OCR). In addition, to the clinical information, the record may include metadata corresponding to one or more of the patient identification parameters. These parameters may include, for example, patient demographic parameters, pharmacy information parameters, diagnosis code parameters, and/or provider encounter parameters. These patient identification parameters may be selected based on their ability to distinguish between patients based on the values assigned thereto. Patient demographic information parameters may be unique or highly correlated with a particular patient. Pharmacy information parameters may not be unique to a particular patient, but if a patient's record includes pages with different pharmacies listed on different pages, then this may be indicative that one of the pages is associated with a different patient. Diagnosis code parameters may be used to identify particular diagnoses that are inconsistent with one another potentially indicating that the diagnoses are associated with different patients. Provider encounter parameters may be used to identify potentially conflicting treatment dates, or unlikely specialty fields or treatment for a particular patient's demographic, for example. The patient demographic parameters may include, but are not limited to, patient name, patient address, patient mobile phone number, patient home phone number, patient work phone number, patient guardian name, patient guardian address, patient guardian mobile phone number, patient guardian home phone number, patient guardian work phone number, patient gender, patient date of birth, patient social security number, patient provider identification number, patient marital status, patient email, patient preferred language, patient race, patient primary care physician name, patient emergency contact name, patient emergency contact phone number, patient employer name, and/or patient employer address. The pharmacy information parameters may include, but are not limited to, pharmacy name, pharmacy address, and/or pharmacy phone number. The patient to whom the record belongs may be termed the first patient and identification values for one or more of the patient identification parameters may be received at block 210. For example, the name, date of birth, gender, home address, mobile phone number, patient provider identification number, and pharmacy name and address may be received for the first patient to which the record belongs. The identification values for the one or more patient identification parameters may be received as part of metadata that accompanies the record. This metadata may include verified patient information for patient to whom the record pertains. The PHI violation detection system 140 may then process the record using a multi-phase analysis system corresponding to an extraction component (block 215), matching component (block 220), linking component (block 225), and assembly component (block 230). These four components may be implemented in multiple ways in accordance with various embodiments of the inventive concept.

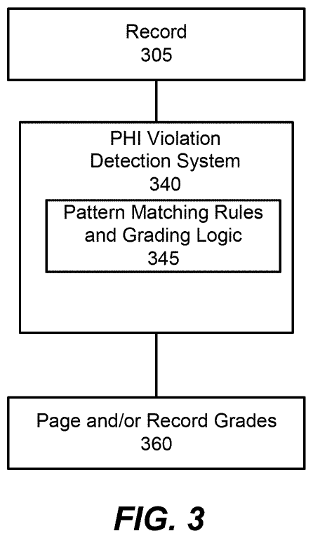

Referring to FIGS. 2 and 3 is a block diagram that illustrates embodiments of the PHI violation detection system in which pattern matching rules and grading logic are used to detect possible PHI violations in a record. Referring to FIG. 3, a claim 305 is provided to the PHI violation detection system 340. The PHI violation detection system 340 is configured with pattern matching rules and grading logic 345 that may be used to implement the extraction component, matching component, linking component, and assembly component. For example, the pattern matching rules 345 may be used to implement the extraction component by processing the record to identify first example instances referencing the patient identification parameters including the values therefor at block 215. In the extraction phase, the record is processed to look for instances of patient identification parameters irrespective of whether the parameter has a value that corresponds to the first patient to whom the record corresponds. Thus, during the extraction phase, the pattern matching rules may extract multiple instances of the patient name parameter in which different names are listed. In accordance with other embodiments of the inventive concept, the PHI violation detection system may use an AI engine in addition to or instead of the pattern matching rules 345 to provide the extraction functionality.

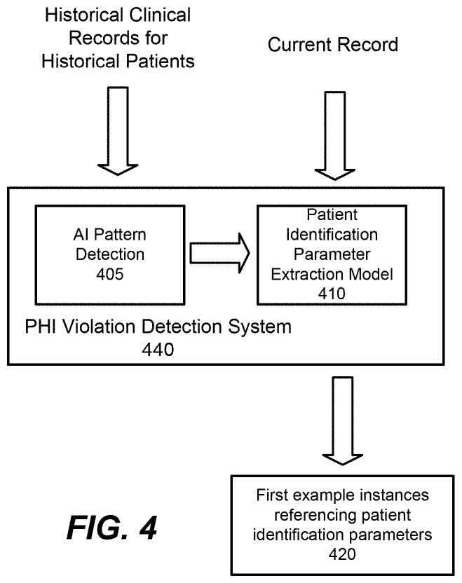

FIG. 4 is a block diagram that illustrates embodiments of the PHI violation detection system in which an AI engine is used to process the record to identify first example instances referencing the patient identification parameters including the values therefor. Various patient identification parameters may appear in various locations in patient records and may not always explicitly identify the parameter as such. For example, the “patient name” parameter may appear in a record with the patient's actual name listed, but without the parameter label “patient name” adjacent thereto. As shown in FIG. 4, the PHI violation detection system 440 includes an AI engine, which may be a machine learning engine comprising an AI pattern detection module 405 and a patient identification parameter extraction model 410. The AI pattern detection module 405 is configured to receive historical clinical records for historical patients, and may learn associations between patient identification parameters and the manners in which the historical clinical information is organized in the records. These associations may, for example, facilitate the ability to identify the presence of a value for a patient identification parameter in circumstances in which the label for the patient identification parameter is missing (e.g., a patient's name is present without the associated label “patient name.”). The AI pattern detection module 405 may then generate a patient identification parameter extraction model 410 based on these learned associations, which can be used to process a current record. The patient identification parameter extraction model 410 may output first example instances in the record that reference the patient identification parameters including the values therefor 420.

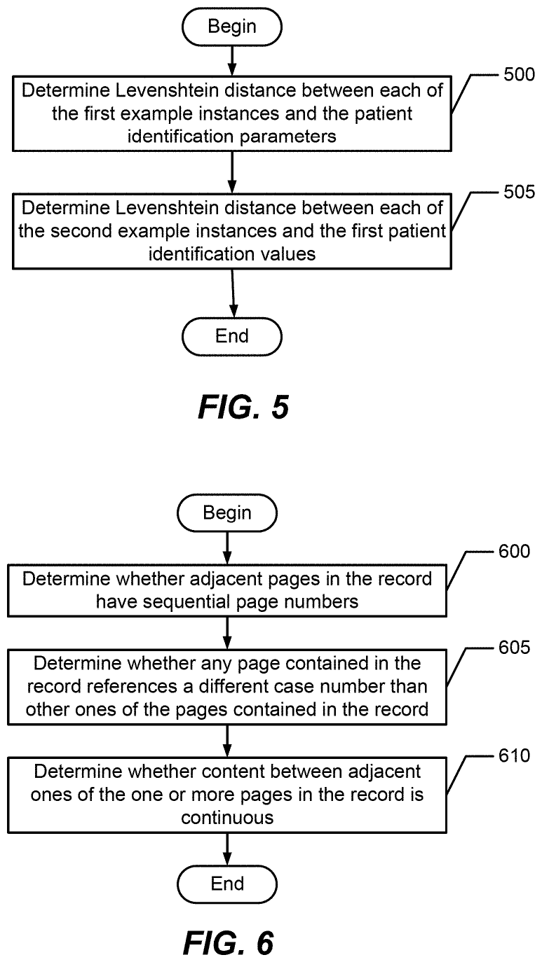

Returning to FIGS. 2 and 3, the pattern matching rules 345 may be used to implement the matching component by processing the record to identify second example instances of the first patient identification values at block 220. In the matching phase, the record is processed to look for instances of patient identification values for one or more of the patient identification parameters that correspond to the first patient to which the record belongs. Thus, the matching phase identifies those instances in the record of patient identification parameter values known to correspond to the first patient to which the record belongs. Various techniques may be used to identify the first example instances of block 215 during the extraction phase and the second example instances of block 220 during the matching phase. For example, referring to FIG. 5, a Levenshtein distance may be determined between each of the first example instances and the patient identification parameters, respectively at block 500 and/or a Levenshtein distance may be determined between each of the second example instances and the first patient identification values, respectively, at block 505. The Levenshtein distance is a string metric that is used to measure the difference between two sequences. For example, in the context of two words, the Levenshtein distance is the minimum number of single-character edits required to change one word into the other. The Levenshtein distance may be determined using a variety of techniques including fuzzy matching, regular expression analysis (REGEX), and/or language modeling. Fuzzy matching assigns a probability to a match between 0.0 and 1.0 based on linguistic and statistical methods instead of just choosing either 1 (true) or 0 (false). REGEX is a standard textual syntax for representing patterns for matching text. Language modeling is the use of various statistical and probabilistic techniques to determine the probability of a given sequence of words occurring in a sentence. Language models analyze bodies of text data to provide a basis for their word predictions. Use of the Levenshtein distance may be illustrated by way of example. A patient record may include a first page with a patient name field value of “Robert Smith” and a second page with a patient name field value of “Rob Smith.” The Levenshtein distance between “Robert Smith” and “Rob Smith” is three. But through use of REGEX, fuzzy matching, and/or language modeling, “Rob” may be determined to be a shortened version of “Robert.” Accordingly, “Rob Smith” may be converted to “Robert Smith” before calculating the Levenshtein distance resulting in a calculated distance value of zero between the patient name field values on the two different pages in the record. Such matching techniques described above may also be used to correct for potential errors in the auto-generated text resulting from an OCR process used to convert a record into text.

The pattern matching rules 345 may evaluate the structure of the record itself to implement the linking component by processing the record to determine whether any of the pages contained therein cannot be semantically linked to another one of the pages at block 225. Example operations of the linking phase are illustrated, for example, in FIG. 6 where one or more determination may be made that may indicate whether a record includes pages associated with different patients. For example, a determination may be made whether adjacent pages in the record have sequential page numbers or other sequential indicia (block 600), a determination may be made whether any page contained in the record references a different case number than other ones of the pages contained in the record (i.e., a case number on one page of a record is compared with a case number on another page of the record providing further confirmation that the pages are linked) (block 605), and/or a determination may be made whether content between adjacent ones of the pages in the record is continuous (block 610). The determination of whether content between adjacent pages is continuous or whether the record includes discontinuities in the content therein may be made using, for example, language modeling.

The grading logic 345 may then take the outputs from the extraction phase, matching phase, and linking phase to assign a grade to each of the pages indicating a degree of confidence that the record does not include clinical information associated with a second patient at block 230. For example, a grade of confirmed may be assigned when the page satisfies a threshold criterion that the page does not include clinical information associated with the second patient. The threshold criterion for a page may, for example, be that the page contains sufficient matched metadata associated with a patient or can be linked to a page that contains sufficient matched metadata for a patient and cannot be linked to an extracted reference to another patient. The threshold criterion for a document or record may be that all pages can be linked to a page with sufficient matched metadata associated with a patient and no page contains extracted references to another patient. The confirmed grade may indicate the page does not need manual review. A grade of suspicious may be assigned when the page does not satisfy the threshold criterion and is predicted to include clinical information associated with the second patient and needs correction. A grade of unknown may be assigned when a prediction whether the page includes clinical information associated with the second patient cannot be made and manual review is recommended. The unknown grade may be assigned, for example, if the PHI violation detection system cannot determine whether the page relates to any patient at all (e.g., the page is a handwritten note with patient identifying information contained thereon). A grade may be assigned to the record based on the grades assigned to each of the pages in the record. The grade may include a summary of the grades for each of the pages in the record.

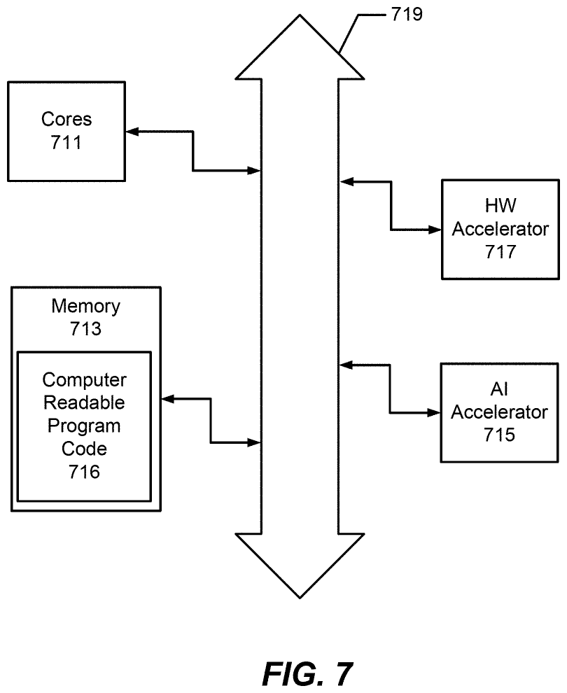

FIG. 7 is a block diagram of a data processing system that may be used to implement the PHI violation detection server 140 of FIG. 1 and/or the PHI violation detection systems 340 and 440 of FIGS. 3 and 4 in accordance with some embodiments of the inventive concept. As shown in FIG. 7, the data processing system may include at least one core 711, a memory 713, an artificial intelligence (AI) accelerator 715, and a hardware (HW) accelerator 717. The at least one core 711, the memory 713, the AI accelerator 715, and the HW accelerator 717 may communicate with each other through a bus 719.

The at least one core 711 may be configured to execute computer program instructions. For example, the at least one core 711 may execute an operating system and/or applications represented by the computer readable program code 716 stored in the memory 713. In some embodiments, the at least one core 711 may be configured to instruct the AI accelerator 715 and/or the HW accelerator 717 to perform operations by executing the instructions and obtain results of the operations from the AI accelerator 715 and/or the HW accelerator 717. In some embodiments, the at least one core 711 may be an ASIP customized for specific purposes and support a dedicated instruction set.

The memory 713 may have an arbitrary structure configured to store data. For example, the memory 713 may include a volatile memory device, such as dynamic random-access memory (DRAM) and static RAM (SRAM), or include a non-volatile memory device, such as flash memory and resistive RAM (RRAM). The at least one core 711, the AI accelerator 815, and the HW accelerator 717 may store data in the memory 713 or read data from the memory 713 through the bus 719.

The AI accelerator 715 may refer to hardware designed for AI applications. In some embodiments, the AI accelerator 715 may include a machine learning engine configured to extract instances of a patient identification parameters including values therefor from a patient clinical record. The AI accelerator 715 may generate output data by processing input data provided from the at least one core 715 and/or the HW accelerator 717 and provide the output data to the at least one core 711 and/or the HW accelerator 717. In some embodiments, the AI accelerator 715 may be programmable and be programmed by the at least one core 711 and/or the HW accelerator 717. The HW accelerator 717 may include hardware designed to perform specific operations at high speed. The HW accelerator 717 may be programmable and be programmed by the at least one core 711.

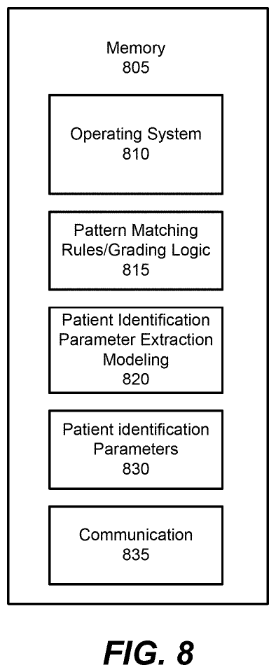

FIG. 8 illustrates a memory 805 that may be used in embodiments of data processing systems, such as the PHI violation detection server 140 of FIG. 1, the PHI violation detection systems 340 and 440 of FIGS. 3 and 4, and the data processing system of FIG. 7, respectively, to facilitate detection of a PHI violation in a patient's clinical record. The memory 805 is representative of the one or more memory devices containing the software and data used for facilitating operations of the PHI violation detection server 140 and the AI/Rules engine module 145 as described herein. The memory 805 may include, but is not limited to, the following types of devices: cache, ROM, PROM, EPROM, EEPROM, flash, SRAM, and DRAM. As shown in FIG. 8, the memory 805 may contain five or more categories of software and/or data: an operating system 810, pattern matching rules/grading logic 915, an AI patient identification parameter extraction modeling module 920, patient identification parameters 930, and a communication module 935. In particular, the operating system 810 may manage the data processing system's software and/or hardware resources and may coordinate execution of programs by the processor.

The pattern matching rules/grading logic module 915 may provide the pattern matching rules and grading logic 345 for detecting possible PHI violations in a patient's clinical record as described above with respect to FIGS. 2 and 3. The AI patient identification parameter extraction modeling module 920 may be configured to perform one or more of the operations described above with respect to the PHI violation detection system 440 of FIG. 4. The patient identification parameters 930 may represent the stored parameters defined at block 200 of FIG. 2 and used in evaluating patient records for PHI violations in accordance with various embodiments of the inventive concept described herein. The communication module 935 may be configured to facilitate communication between the PHI violation detection server 140 of FIG. 1 and/or the PHI violation detection systems 340 and 440 of FIGS. 3 and 4 and entities, such as providers, insurance claim payors, clinical record auditing entities, data entry entities, and the like that may use embodiments of the inventive concept to evaluate patient records for PHI violations.

Although FIGS. 7 and 8 illustrate hardware/software architectures that may be used in data processing systems, such as the PHI violation detection server 140 of FIG. 1, the PHI violation detection systems 340 and 440 of FIGS. 3 and 4, and the data processing system of FIG. 7, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-8 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the health care facility interface server 130 of FIG. 1, the PHI violation detection server 140 of FIG. 1, the PHI violation detection systems 340 and 440 of FIGS. 3 and 4, and the data processing system of FIG. 7 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.” The functionality provided by the health care facility interface server 130 and the PHI violation detection server 140 may be merged into a single server or maintained as separate servers in accordance with different embodiments of the inventive concept.

The data processing apparatus described herein with respect to FIGS. 1-8 may be used to facilitate detection of PHI violations in patient clinical records according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 805 when coupled to a processor includes computer readable program code that, when executed by the processor, causes the processor to perform operations including one or more of the operations described herein with respect to FIGS. 1-6.

Some embodiments of the inventive concept may provide a PHI violation detection system that can be used by entities, such as providers, payors (e.g., insurers), clinical record auditors, and/or data entry personnel, to process patient clinical records and identify pages in the records that can be confirmed as not needing manual review to check for possible PHI violations while also identifying those pages that likely need correction and/or manual review to ensure the record does not include pages associated with different patients thereby causing a PHI violation. This may improve efficiency and costs associated with the review of patient records for PHI violations as most clinical record pages may be confirmed using the automated PHI violation detection system as not requiring manual review leaving a relatively small portion to be inspected manually.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a record comprising at least one page and containing clinical information associated with a first patient;
receiving respective first patient identification values for one or more patient identification parameters corresponding to the first patient;
automatically processing the record to identify first example instances referencing the patient identification parameters including values therefor;
automatically processing the record to identify second example instances of the first patient identification values;
automatically processing the record to determine whether any of the at least one page contained therein cannot be semantically linked to another one of the at least one page in the record; and
assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.

2. The method of claim 1, further comprising:
converting the record into a text record;
wherein automatically processing the record to identify first example instances, automatically processing the record to identify second example instances, and automatically processing the record to determine whether any of the at least one page contained therein cannot be semantically linked to another one of the at least one page in the record, comprises:
processing the text record to identify first example instances, processing the text record to identify second example instances, and processing the text record to determine whether any of the at least one page contained therein cannot be semantically linked to another one of the at least one page in the text record.

3. The method of claim 2, wherein converting the record into the text record comprises converting the record into the text record using optical character recognition (OCR).

4. The method of claim 1, wherein the patient identification parameters comprise: patient demographic parameters, pharmacy information parameters, diagnosis code parameters, and provider encounter parameters.

5. The method of claim 4, wherein the patient demographic parameters comprise: patient name, patient address, patient mobile phone number, patient home phone number, patient work phone number, patient guardian name, patient guardian address, patient guardian mobile phone number, patient guardian home phone number, patient guardian work phone number, patient gender, patient date of birth, patient social security number, patient provider identification number, patient marital status, patient email, patient preferred language, patient race, patient primary care physician name, patient emergency contact name, patient emergency contact phone number, patient employer name, and/or patient employer address.

6. The method of claim 4, wherein the pharmacy information parameters comprise: pharmacy name, pharmacy address, and/or pharmacy phone number.

7. The method of claim 4, wherein the provider encounter parameters comprise:
encounter date, name of medical professional, and/or identification of service performed.

8. The method of claim 1, wherein automatically processing the record to identify the first example instances referencing the patient identification parameters including the values therefor comprises:
determining a Levenshtein distance between each of the first example instances referencing the patient identification parameters and the patient identification parameters, respectively.

9. The method of claim 8, wherein determining the Levenshtein distance comprises:
determining the Levenshtein distance using fuzzy matching, regular expression analysis, and/or language modeling.

10. The method of claim 1, wherein automatically processing the record to identify the second example instances of the first patient identification values comprises:
determining a Levenshtein distance between each of the second example instances of the first patient identification values and the first patient identification values, respectively.

11. The method of claim 10, wherein determining the Levenshtein distance comprises:
determining the Levenshtein distance using fuzzy matching, regular expression analysis, and/or language modeling.

12. The method of claim 1, wherein automatically processing the record to identify the first example instances referencing the patient identification parameters including the values therefor comprises:
generating a patient identification parameter extraction model based on historical records containing historical clinical information for patients in which associations are learned between the patient identification parameters and manners in which the historical clinical information is organized in the historical records.

13. The method of claim 12, wherein generating the patient identification parameter extraction model comprises:
using an Artificial Intelligence (AI) system to learn the associations between the patient identification parameters and the manners in which the historical clinical information is organized in the historical records.

14. The method of claim 1, wherein automatically processing the record to determine whether any of the at least one page contained therein cannot be semantically linked to another one of the at least one page in the record comprises:
determining whether adjacent pages in the record have sequential numbers;
determining whether any of the at least one page contained in the record references a different case number than other ones of the at least one page contained in the record; and/or
determining whether content between adjacent ones of the at least one page in the record is continuous.

15. The method of claim 1, wherein assigning the grade to each of the at least one page indicating the degree of confidence that the record does not include clinical information associated with the second patient comprises:
assigning the grade of confirmed when the page satisfies a threshold criterion that the page does not include clinical information associated with the second patient indicating the page does not need manual review;
assigning the grade of suspicious when the page does not satisfy the threshold criterion and is predicted to include clinical information associated with the second patient and needs correction; and
assigning the grade of unknown when a prediction whether the page includes clinical information associated with the second patient cannot be made and manual review is recommended.

16. The method of claim 1, wherein assigning the grade to each of the at least one page indicating the degree of confidence that the record does not include clinical information associated with the second patient comprises:
assigning a grade to the record based on the grades assigned to each of the at least one page indicating a degree of confidence that the record does not include clinical information associated with the second patient.

17. A system, comprising:
a processor; and
a memory coupled to the processor and comprising computer readable program code embodied in the memory that is executable by the processor to perform operations comprising:
receiving a record comprising at least one page and containing clinical information associated with a first patient;
receiving respective first patient identification values for one or more patient identification parameters corresponding to the first patient;
automatically processing the record to identify first example instances referencing the patient identification parameters including values therefor;
automatically processing the record to identify second example instances of the first patient identification values;
automatically processing the record to determine whether any of the at least one page contained therein cannot be semantically linked to another one of the at least one page in the record; and
assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.

18. The system of claim 17, wherein automatically processing the record to identify the first example instances referencing the patient identification parameters including the values therefor comprises:
determining a Levenshtein distance between each of the first example instances referencing the patient identification parameters and the patient identification parameters, respectively; and
wherein automatically processing the record to identify the second example instances of the first patient identification values comprises:
determining a Levenshtein distance between each of the second example instances of the first patient identification values and the first patient identification values, respectively.

19. The system of claim 17, wherein assigning the grade to each of the at least one page indicating the degree of confidence that the record does not include clinical information associated with the second patient comprises:
assigning the grade of confirmed when the page satisfies a threshold criterion that the page does not include clinical information associated with the second patient indicating the page does not need manual review;
assigning the grade of suspicious when the page does not satisfy the threshold criterion and is predicted to include clinical information associated with the second patient and needs correction; and
assigning the grade of unknown when a prediction whether the page includes clinical information associated with the second patient cannot be made and manual review is recommended.

20. A computer program product, comprising:
a non-transitory computer readable storage medium comprising computer readable program code embodied in the medium that is executable by a processor to perform operations comprising:
receiving a record comprising at least one page and containing clinical information associated with a first patient;
receiving respective first patient identification values for one or more patient identification parameters corresponding to the first patient;
automatically processing the record to identify first example instances referencing the patient identification parameters including values therefor;
automatically processing the record to identify second example instances of the first patient identification values;
automatically processing the record to determine whether any of the at least one page contained therein cannot be semantically linked to another one of the at least one page in the record; and
assigning a grade to each of the at least one page indicating a degree of confidence that the record does not include clinical information associated with a second patient based on the first example instances, the second example instances, and the determination whether any of the at least one page contained therein cannot be semantically linked to another one of the at least one page in the record.
